(19) United States
(12) Patent Application Publication
(10) Pub. No.: US 2011/0274629 A1
(43) Pub. Date: Nov. 10, 2011

(54) Natural Polymer Blends for Use in Personal Care Products

(75) Inventors: Wei Li, San Diego, CA (US); Zhi-Fa Yang, San Diego, CA (US); Todd Talashek, San Diego, CA (US); Anju Bansal, San Diego, CA (US)

(73) Assignee: CP Kelco U.S., Inc., Atlanta, GA (US)

(21) Appl. No.: 12/773,440

(22) Filed: May 4, 2010

(51) Int. Cl. A61K 8/73; A61K 31/715; A61Q 11/00; A61Q 5/02; A61Q 5/06; A61Q 5/12; A61K 31/723; A61P 31/04

(52) U.S. Cl. 424/49; 514/54; 424/70.13

(57) ABSTRACT

Embodiments of the present description provide a blend for use in a personal care product having a modified xanthan gum and a galactomannan, wherein the modified xanthan gum is a non-pyruvylated xanthan gum, and the modified xanthan gum and the galactomannan are present in the blend in a ratio from about 1:99 to about 99:1 by weight. Also provided in embodiments of the present description are personal care products having blends of modified xanthan gums and galactomannans, methods for product formulation, and use thereof.

### BACKGROUND OF THE INVENTION

Personal care products sometimes require the addition of thickening agents to improve properties such as viscosity and skin feel. A good thickening agent should provide a moderate-to-high viscosity, a good shape retention, and an aesthetically pleasing “non-tacky” feel. Thickening agents desirably should be tolerant of salts, metallic cations, and other electrolytes that are sometimes found in personal care products. Additionally, thickening agents desirably should not disturb the overall clarity and smooth, textural qualities of personal care product formulations upon inclusion. Finally, a thickening agent desirably should be non-toxic if used for personal care applications.

One common ingredient used in formulating personal care products is the thickening agent Carbopol® (Lubrizol Corporation, Cleveland, Ohio). Carbopol®, which is used generically to refer to several Carbopol® products including Carbopol® Ultrez 20 polymer, Carbopol® Ultrez 21 polymer, and Carbopol® ETD 2020 polymer, is a synthetic C10-30 alkyl acrylate cross-polymer. Carbopol® polymers may be used in a variety of personal care products, including shampoos, body washes, bath gels, lotions, hair and skin gels, creams, hand sanitizers, sunscreen lotions, and aloe vera gels. Although Carbopol® is successfully used in many personal care products, Carbopol® is a non-natural synthetic acrylate polymer.

In today's consumer markets, there is a greater demand for natural, non-synthetic additives for use in personal care product formulations. Existing natural thickening agents, such as xanthan gum or galactomannans, however, have been unable to achieve the same desirable properties as synthetic thickening agents.

Xanthan gum is a microbial polysaccharide produced by a pure culture of aerobic submerged fermentation of Xanthomonas campestris. Native xanthan gum is comprised of a 1,4-linked D-glucose backbone with trisaccharide side chains on alternating anhydroglucose units. The side chains are comprised of a glucuronic acid residue between two mannose units. Approximately 50% of the terminal mannose molecules carry a pyruvic acid residue. Xanthan gum has a pseudoplastic or shear-thinning behavior characterized by a decrease in apparent viscosity in response to an increase in shear rate. Its ability to hydrate in either hot or cold water allows xanthan gum to be used in a broad variety of applications, including pharmaceuticals, household products, foods, and personal care products.

Galactomannans are a group of neutral polysaccharides naturally occurring in the seeds of some species of the Leguminosae family. Galactomannans are comprised of a mannose backbone with galactose side groups. More particularly, the galactomannan molecule comprises a 1,4-linked β-mannopyranose backbone with branchpoints of α-D-galactose from the C6 positions of the mannose backbone. The ratio of mannose to galactose varies according to the species of the plant from which the galactomannan is derived. Common galactomannans, and their corresponding ratios of mannose to galactose, include fenugreek gum (1:1), guar gum (2:1), tara gum (3:1), locust bean gum (4:1), and cassia gum (5:1). Galactomannans are commonly used in many applications as rheology modifiers, and have been shown to behave synergistically in combination with other rheological modifiers.

Synergistic interactions between galactomannans and native xanthan gum are well documented. At high total gum concentrations, aqueous solutions of galactomannans and xanthan gum will form elastic gels. The lower the level of galactose substitution on the mannose backbone, the greater the synergy of the galactomannan with xanthan gum. For example, locust bean gum (which contains about 20% galactose) forms strong elastic gels in combination with equal amounts of xanthan gum in aqueous solutions. Guar gum (which contains about 35% galactose), instead forms weaker elastic gels when combined with xanthan gum in aqueous solutions.

These synergistic effects between xanthan gum and galactomannans have been reported in numerous applications; however, xanthan gum-galactomannan blends have been unable to provide the same desirable rheological properties as the synthetic thickening agent Carbopol®. Accordingly, there still exists a need for natural thickening agents to impart Carbopol®-like thickening properties to personal care products. Natural thickening agents are particularly desirable in personal care products because they may lack undesirable properties, such as a slimy skin feel and a difficulty in washing out, that have been observed with the use of personal care products containing synthetic polymers. Additionally, natural thickening agents may satisfy consumer demand for all-natural personal care products.

### SUMMARY OF THE INVENTION

Embodiments of the present description comprise a blend for use in personal care products comprising a modified xanthan gum and a galactomannan, wherein the modified xanthan gum comprises a non-pyruvylated xanthan gum, and wherein the modified xanthan gum and the galactomannan are present in the blend in a ratio from about 1:99 to about 99:1 by weight.

Other embodiments of the present description comprise personal care products comprising a blend of a modified xanthan gum and a galactomannan, wherein the modified xanthan gum comprises a non-pyruvylated xanthan gum, wherein the modified xanthan gum and the galactomannan are present in the blend in a ratio from about 1:99 to about 99:1 by weight, and wherein the blend is present in the personal care product in an amount from about 0.1% to about 4.0% by weight of the personal care product.

Still other embodiments of the present description comprise methods for preparing blends for use in personal care products comprising combining a modified xanthan gum with a galactomannan to form a blend, wherein the modified xanthan gum comprises a non-pyruvylated xanthan gum and wherein the modified xanthan gum and the galactomannan are present in the blend in a ratio from about 1:99 to about 99:1 by weight.

Additional aspects will be set forth in part in the description which follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

### DETAILED DESCRIPTION

The present application addresses the above-described needs by providing natural blends that are suitable for use as thickening agents in personal care product formulations. In particular, the blends for use in personal care products comprise a blend of a modified xanthan gum and a galactomannan, wherein the modified xanthan gum comprises a non-pyruvylated xanthan gum, and wherein the modified xanthan gum and the galactomannan are present in the blend in a ratio from about 1:99 to about 99:1 by weight. Also provided herein is a personal care product comprising a blend of a modified xanthan gum and a galactomannan, methods for product formulation, and use thereof.

Although the use of a native xanthan gum with a galactomannan is known, it was not known or predicted that the use of a blend comprising a modified xanthan gum and a galactomannan would impart significantly improved rheological properties to personal care products. Applicants surprisingly discovered that a blend of a modified xanthan gum and a galactomannan is capable of imparting rheological properties to personal care products similar to that of synthetic acrylate polymers. In particular, use of a blend of a modified xanthan gum and a galactomannan in a personal care product was found to impart properties to personal care products that are similar to personal care products comprising Carbopol® and that are significantly improved over personal care products comprising a native xanthan gum-galactomannan blend, a non-pyruvylated xanthan gum alone, or a galactomannan alone.

Personal care products prepared using high concentrations of native xanthan gum-galactomannan blends will not flow and are unable to be readily picked up due to the formation of strong, elastic gels that are not easily deformed. Personal care products prepared using lower concentrations of native xanthan gum-galactomannan blends, however, have insufficient shape retention. Similarly, use of non-pyruvylated xanthan gum alone or a galactomannan alone in personal care products also fails to provide the desired shape retention to the personal care product. The use of modified xanthan gum-galactomannan blends provided in embodiments of the present description, however, are capable of imparting to the personal care products a desirable viscosity, shape retention, and pick-up, while maintaining smoothness and providing excellent clarity. Not wishing to be bound by any theory, it is believed that the combination of a modified xanthan gum and a galactomannan results in a weaker synergistic interaction that surprisingly gives the desired properties of shape retention, pick up, smoothness, and hardness. These rheological properties are determined, at least in part, by the ratio of the modified xanthan gum to the galactomannan present in the blend.

Desirably, the blends of the modified xanthan gum and galactomannan are capable of forming a yield stress fluid. As used herein, the term “yield stress fluid” refers to a composition characterized as having good shape retention, good deformability (i.e., low hardness), and good flow properties.

Whether a composition has the desired shape retention and hardness is evaluated using Texture Profile Analysis (TPA), which is conventionally used for characterizing gels (see http://www.texturetechnologies.com/texture_profile_analysis.html for references using TPA). The height of the composition after de-molding is indicative of the composition's ability to retain its initial shape (i.e., shape retention) while the ability of a composition to sustain resistance to a compression force is indicative of the composition's hardness. Methods for measuring the hardness and shape retention are known to those skilled in the art and are described in more detail below.

Whether a composition has the desired flow properties is evaluated by measuring the shear viscosities of the blends in aqueous solutions. These properties also can be used to further characterize a composition's shape retention and pick up. A high value of the low shear viscosity is indicative of a composition having a better shape retention. A high value of the high shear viscosity is indicative of a composition having a better pick up. Desirably, the compositions provided herein have high values for both the low shear viscosity and the high shear viscosity.

### A. Blends of Modified Xanthan Gum and a Galactomannan

In one embodiment, a blend for use in a personal care product is provided comprising a modified xanthan gum and a galactomannan, wherein the modified xanthan gum comprises a non-pyruvylated xanthan gum, and wherein the modified xanthan gum and the galactomannan are present in the blend in a ratio from about 1:99 to about 99:1 by weight.

Modified Xanthan Gums

As used herein, a “non-pyruvylated xanthan gum” (NPX) is synonymous with a de-pyruvylated xanthan gum and comprises a modified xanthan gum having substantially no pyruvate. A modified xanthan gum having “substantially no pyruvate,” as used herein, means a modified xanthan gum having a pyruvate content of up to about 0.5% by weight.

The modified xanthan gum can be prepared by fermentation of any strain capable of producing a modified xanthan gum having substantially no pyruvate content when compared to that of a native xanthan gum. For example, a suitable modified xanthan gum may be produced using the non-pyruvate Xanthomonas campestris (See U.S. Pat. No. 6,316,614 and Biotechnol. Prog. 1990, 6, 182-187 for descriptions on a similar strain). Other examples of strains capable of producing non-pyruvylated xanthan gum include ATCC deposits 53472 and 67344. (See U.S. Pat. No. 6,316,614). The modified xanthan gum may be recovered from the fermentation broth and treated using methods known to those skilled in the art (e.g., clarification, pasteurization, precipitation, drying, and milling).

Alternatively, pyruvate substituents may be removed from a native xanthan gum by chemical treatment. (See, e.g., Carbohydrate Polymers, 1983, 3, 23-28).

Galactomannans

A galactomannan, as used herein, comprises any naturally occurring galactomannan that is commercially available. For example, a galactomannan typically is obtained as a naturally occurring material such as from seeds or beans of plants. In one embodiment, the galactomannan is fenugreek gum, guar gum, tara gum, locust bean gum, cassia gum, or combinations thereof. The galactomannan also can comprise modified galactomannans (i.e., a galactomannan in which the galactose substitutions have been modified).

The galactomannans provided herein can be characterized by the degree of galactose substitutions present on the mannose backbone. Galactomannans having a low degree of galactose substitutions, as used herein, comprise a mannose to galactose ratio from about 2.5:1 to about 5:1, non-limiting examples of which include tara gum, locust bean gum, and cassia gum. Galactomannans having a high degree of galactose substitutions, as used herein, comprise a mannose to galactose ratio from about 1:1 to about 2.5:1, non-limiting examples of which include fenugreek gum and guar gum.

Modified Xanthan Gum-Galactomannan Blends

Blends comprising a modified xanthan gum and a galactomannan provided herein can comprise the modified xanthan gum and the galactomannan in any ratio that is effective to impart the desired rheological properties to the blend and to the personal care products to which the blend is added. In one embodiment, the modified xanthan gum and the galactomannan are present in the blend in a ratio from about 1:99 to about 99:1 by weight.

Surprisingly, the ratios of the modified xanthan gum to the galactomannan depend upon the type of galactomannan (i.e., the degree of galactose substitution) being used in the blend. Galactomannans having a low degree of galactose substitution desirably are used in blends having either high ratios of the modified xanthan gum to the galactomannan or low ratios of the modified xanthan gum to the galactomannan. Galactomannans having a high degree of galactose substitution desirably are used in blends having intermediate ratios of the modified gum to the galactomannan.

In one aspect, the blends comprise a modified xanthan gum and a galactomannan having a low degree of galactose substitution present in the blend in a ratio from about 4:1 to about 99:1 by weight or in a ratio from about 1:4 to about 1:99 by weight, about 10:1 to about 99:1 by weight or in a ratio from about 1:10 to about 1:99 by weight, about 30:1 to about 99:1 by weight or about 1:30 to about 1:99 by weight, about 50:1 to about 99:1 by weight or about 1:50 to about 1:99 by weight, or about 80:1 to about 99:1 by weight, or about 1:80 to about 1:99 by weight.

In another aspect, the blends comprise a modified xanthan gum and a galactomannan having a high degree of galactose substitution present in the blend in a ratio from about 1:9 to about 9:1 by weight, about 1:5 to about 5:1 by weight, about 1:3 to about 3:1 by weight, or about 1:2 to about 2:1 by weight.

Those of ordinary skill in the art will appreciate that the blends may be prepared using any suitable method of mixing the modified xanthan gum and galactomannan. For example, in one embodiment the blend is prepared by combining a modified xanthan gum with a galactomannan. In embodiments, the modified xanthan gum and the galactomannan may be combined as individual dry powders and mixed into a homogenous dry powder blend. Methods of mixing are well known to those skilled in the art, non-limiting examples of which include stirring, shaking, and agitation. In addition to dry blending, the individual components (i.e., the modified xanthan gum and the galactomannan) may be added to the personal care formulation separately. The modified xanthan gum and the galactomannan also may be blended together in a solution, and optionally dried, before adding the blend to the personal care product formulation.

Rheological Properties of Modified Xanthan Gum-Galactomannan Blends

In particular embodiments, the blends of the modified xanthan gum and galactomannan are characterized as having desired rheological properties based on the viscosity profile of the blends. For example, in one aspect a blend is characterized as having a desirable viscosity profile as measured using a Brookfield LV-type viscometer using spindle #64. In one embodiment, a desirable viscosity profile comprises a low shear viscosity in water (measured at about 0.6 rpm and ambient conditions) of between about 100,000 cP and about 300,000 cP and a high shear viscosity in water (measured at about 12 rpm and ambient conditions) of between about 25,000 cP and about 40,000 cP. In still another embodiment, a desirable viscosity profile comprises a low shear viscosity of between about 100,000 cP and about 200,000 cP and a high shear viscosity of between about 30,000 cP and about 40,000 cP. In still a further embodiment, a desirable viscosity profile comprises a low shear viscosity of between about 100,000 cP and about 150,000 cP and a high shear viscosity between about 35,000 cP and about 40,000 cP.

In particular embodiments, the blends of the modified xanthan gum and galactomannan are characterized as having desired rheological properties based on the hardness of the blends. The term “hardness,” as described above, characterizes a composition's ability to sustain resistance to a compression force. The blends of the modified xanthan gum and galactomannan desirably have a low value of hardness while still having good shape retention. In one embodiment, the hardness is measured by the resistance force detected by the probe tip of a Texture Profile Analyzer (TPA) during a Compression Test. In one embodiment, a blend is characterized as having desirable textural properties when it has a hardness from about 800 Pa to about 1100 Pa, from about 800 Pa to about 1000 Pa, or from about 800 Pa to about 900 Pa.

In particular embodiments, the blends of the modified xanthan gum and galactomannan are further characterized as having desired rheological properties based on the shape retention of the blends. The blend is prepared and hydrated with water and poured into the acrylic ring molds (internal diameter: 29 mm; height: 12 mm), subsequently de-molded from the acrylic ring mold, and the height of the composition is immediately measured. (See Examples below). The greater the height (relative to the height of the acrylic ring mold), the better the shape retention of the composition. For example, in one aspect a composition is characterized as having good shape retention when it retains at least 60% of its height, at least 70% of its height, at least 80% of its height, at least 90% of its height, or at least 95% of its height.

### B. Personal Care Products

Also provided in embodiments of the present description are personal care products comprising the blends of the modified xanthan gum and galactomannan in an amount effective to impart desired rheological and sensory properties to the personal care product. Non-limiting examples of personal care products to which the blends may be added include a styling gel, a moisturizer, a lotion, a deodorant, a toothpaste, a body wash, a bath gel, a body gel, a hand sanitizer, a shampoo, a conditioner, or combinations thereof.

Those skilled in the art will appreciate that the rheological properties of the personal care product may be modified, in part, by modifying the amount of the blend present in the personal care product. In one embodiment, the blend of the modified xanthan gum and galactomannan is present in the personal care product in an amount from about 0.1% to about 4.0% by weight of the personal care product. For example, in embodiments the blend may be present in the personal care product in an amount from about 0.1% to about 3.0% by weight, in an amount from about 0.1% to about 2.0% by weight, or in an amount from about 0.1% to about 1.0% by weight.

The personal care products of the present description may further comprise one or more additional components known for use in personal care products, provided that the additional components are physically and chemically compatible with the blends provided herein, or do not otherwise unduly impair product stability, aesthetics, or performance. Individual amounts of such additional components may range from about 0.001% to about 95% by weight of the personal care product. The additional components may be water soluble (i.e., soluble in water at 25° C.) or water insoluble (not soluble in water at 25° C.). Non-limiting examples of such additional components include conditioning agents, silicones, hydrocarbon oils, fatty esters, particles, suspending agents, paraffinic hydrocarbons, propellants, viscosity modifiers, dyes, non-volatile solvents or diluents, pearlescent aids, foam boosters, ionic and nonionic surfactants, ionic and non-ionic co-surfactants, pH adjusting agents, perfumes, preservatives, chelating agents, pediculides, proteins, skin active ingredients, sunscreens, UV absorbers, vitamins, fatty acids, and fatty oils.

The personal care products provided in embodiments of the present description may be characterized by the same rheological properties used to characterize the rheological properties of the blends above. For example, the pick up and shape retention of the personal care product may be characterized by the viscosity of the personal care product and the textural properties may be characterized by the hardness or shape retention of the personal care product. Desirably, the personal care products provided in embodiments of the present description are characterized as having one or more rheological properties that are substantially similar to the rheological properties of a personal care product comprising a traditional thickening agent, such as Carbopol®. The rheological properties of the personal care products are considered to be “substantially similar” when the average viscosity, hardness, and/or shape retention of the personal care product prepared using the blends in the present description are at least 70% of the average viscosity, hardness, and/or shape retention of personal care products prepared using a traditional thickening agent. In still other embodiments, the rheological properties are considered to be substantially similar when they are within at least 80%, within at least 90%, or within at least 95% of the average viscosity, hardness, and/or shape retention of the personal care products prepared using a traditional thickening agent.

The personal care products provided in embodiments of the present description also may be characterized as having sensory properties that are substantially similar to those personal care products prepared using traditional thickening agents. Methods for characterizing the sensory properties of personal care products are well known to those skilled in the art and include the evaluation of the personal care products by trained panelists of various sensory properties (i.e., clarity, pick-up, smoothness, rub-out, wash-out, and after-feel). The sensory properties of the personal care product are considered to be “substantially similar” when the average sensory property score of the personal care products prepared using the blends in the present description are at least 80% the average sensory property score of personal care products prepared using a traditional thickening agent. In still other embodiments, the sensory properties are considered to be substantially similar when they are within at least 85%, within at least 90%, or within at least 95% of the average sensory property score of the personal care products prepared using a traditional thickening agent.

The following examples illustrate specific embodiments of the present description, but are not intended to be limiting thereof. Other modifications can be undertaken by the skilled artisan without departing from the spirit and the scope of this description.

### EXAMPLES

### Preparation of Modified Xanthan Gum

A non-pyruvylated xanthan gum (NPX) was prepared using a non-pyruvate X. campestris strain (e.g., U.S. Pat. No. 6,316,614 for a similar type strain) in a 1200 gallon fermentor, treated with a standard enzyme clarification process, and recovered by conventional pasteurization, precipitation, drying, and milling steps. The NPX had a pyruvate content of less than 0.5% by weight.

### Preparation of Blends

A homogenous dry powder blend was prepared using the modified xanthan gum described above and either purified guar gum (Ajinomoto RG 100® from Ajinomoto USA, Inc., Fort Lee, N.J.) or clarified locust bean gum (GENU GUM RL-200Z® from CP Kelco, USA). Representative blends comprising a modified xanthan gum and a galactomannan of the present description are set forth in the table below.

| | NPX | Galactomannan | NPX: |
|---|---|---|---|
| Blend | (% by weight) | (% by weight) | Galactomannan |
| Blend 1 | 50% | Guar gum | 1:1 |
| | | (50%) | |
| Blend 2 | 33% | Guar gum | 1:2 |
| | | (67%) | |
| Blend 3 | 25% | Guar gum | 1:3 |
| | | (75%) | |
| Blend 4 | 97.5% | Locust bean gum | 39:1 |
| | | (2.5%) | |
| Blend 5 | 2% | Locust bean gum | 1:49 |
| | | (98%) | |
| Blend 6 | 99% | Locust bean gum | 99:1 |
| | | (1%) | |

A control blend comprising a 1:1 mixture of a native xanthan gum (KELTROL T® from CP Kelco, USA) and purified guar gum (Ajinomoto RG 100® from Ajinomoto USA, Inc., Fort Lee, N.J.) was prepared in a similar fashion.

The rheological properties (i.e., viscosity, hardness, and shape retention) of solutions of the blends were measured using a 1.5 wt % solution of a blend in deionized water. The solutions were stirred with a four-prong propeller mixer at 500-2000 rpm for two hours and rheological measurements were performed no sooner than 24 hours after the preparation of the solution.

The viscosity of solutions of the blends was measured with a Brookfield LV-type viscometer using spindle #64 after about one minute of rotation. Both the low shear viscosity (at about 0.6 rpm) and the high shear viscosity (at about 12 rpm) were measured and are set forth in the table below.

The hardness and shape retention for each blend solution were measured using a TA-X2.Ti texture analyzer (Texture Technologies, Scarsdale, N.Y.). The shape retention and the hardness of the solutions of the blends were measured by TPA. Immediately after measuring the viscosity, the solutions of the blends were poured into 2 acrylic ring molds (diameter: 29 mm; height: 12 mm). Each molded sample was subsequently de-molded onto a measuring plate and both the height and the hardness of the sample was measured by a compression test. These results are set forth in the table below.

| | Hardness | Viscosity @ | Viscosity @ | Height | Shape Retention |
|---|---|---|---|---|---|
| Sample | (Pa) | 0.6 rpm (cP) | 12 rpm (cP) | (mm) | (% Height) |
| 0.3% Carbopol ® | 815.20 | 147000 | 36250 | 10.60 | 88.3% |
| Ultrez 21 | | | | | |
| [control] | | | | | |
| 1.5% NPX | 425.99 | 110000 | 13000 | 7.96 | 66.3% |
| [control] | | | | | |
| 2% NPX | 619.20 | 168000 | 18950 | 9.62 | 80.2% |
| [control] | | | | | |
| 1.5% guar gum | 880.23 | 13000 | 8900 | 3.75 | 31.3% |
| [control] | | | | | |
| 2% guar gum | 1899.64 | 4300 | 25600 | 4.46 | 37.2% |
| [control] | | | | | |
| native xanthan/ | 2921.91 | 701000 | >50000 | 7.77 | 64.8% |
| guar gum | | | | | |
| [control] | | | | | |
| Blend 1 | 1090.31 | 269000 | 28250 | 8.10 | 67.5% |
| Blend 2 | 1134.92 | 295000 | 33400 | 8.10 | 67.5% |
| Blend 3 | 1034.49 | 252000 | 30300 | 6.86 | 57.2% |
| Blend 4 | 984.89 | 113000 | 11550 | 8.5 | 70.8% |
| Blend 5 | 1371.32 | 164000 | 41741 | 5.67 | 47.3% |
| Blend 6 | 939.10 | 244000 | 23145 | 9.07 | 75.6% |

As the foregoing illustrates, the solutions of the blends of a modified xanthan gum and a galactomannan provided a more desirable (relative to Carbopol®) range of hardness, low shear viscosity, high shear viscosity, and shape retention as compared to solutions having either NPX alone, guar gum alone, or a mixture of a native xanthan gum and guar gum. Notably, the solutions of the blends of the modified xanthan gum and a galactomannan showed rheological properties that were substantially similar to the solutions of Carbopol® over multiple parameters.

### Personal Care Product Formulations

A homogenous dry powder blend (Blend 3) was added to deionized water and stirred with a four-prong propeller mixer at 500-2000 rpm until homogenous. At five minute intervals, one or more additional ingredients were added to the stirring solution using the representative formulations described below. The final formulation was stirred continuously until homogenous.

| | Soft-Hold | Aloe Vera Gel | Hand and |
|---|---|---|---|
| Ingredient | Styling Gel | Moisturizer | Body Lotion |
| Water (de-ionized) | 92.9% | 94.2% | 83.6% |
| Blend 3 | 3.8% | 1.8% | 1.0% |
| Fragrance | 2.2% | — | — |
| Polysorbate 20 | 0.1% | — | — |
| Preservative | 0.4% | 0.3% | 0.3% |
| Fixate G-100 | 3.8% | — | — |
| SilSense Copolyol | — | 1.0% | — |
| Disodium EDTA | — | 0.2% | 0.1% |
| Aloe Vera Gel 10X | — | 2.5% | — |
| Propylene glycol | — | — | 0.8% |
| Glycerin | — | — | 5% |
| Mineral oil | — | — | 4.0% |
| Stearic acid | — | — | 2.0% |
| Glycol Stearate | — | — | 1.5% |
| Cetyl acetate | — | — | 0.5% |
| Glycol Stearate SE | — | — | 0.5% |
| Cetyl alcohol | — | — | 0.2% |
| Dimethicone | — | — | 0.5% |

Controls of the personal care products were prepared in a similar manner using Carbopol® Ultrez 21 in place of Blend 3. Additionally, Pantene® Curls Gel, Vaseline® Aloe Fresh Moisturizing Lotion, and Olay® Active Hydrating Beauty Fluid were used as controls without further modification of the commercially-available products.

The rheological properties (i.e., viscosity, hardness, and shape retention) of the personal care products were characterized using the same methods of measuring the viscosity, hardness, and shape retention as were used to characterize the blends. The results are summarized in the table below.

| | | | | | |
|---|---|---|---|---|---|
| | Hardness | Viscosity @ | Viscosity @ | Height | Shape |
| Sample | (Pa) | 0.6 rpm (cP) | 12 rpm (cP) | (mm) | Retention (% |

Soft-Hold Styling

| | | | | | |
|---|---|---|---|---|---|
| Blend 3 | 1450.14 | 406000 | 49300 | 8.86 | 73.8% |
| Carbopol | 823.65 | 530000 | 49350 | 10.44 | 87% |
| Pantene Curls Gel | 811.70 | 454000 | 46300 | 10.82 | 90.2% |

Aloe Vera Gel
Moisturizer

| | | | | | |
|---|---|---|---|---|---|
| Blend 3 | 1416.08 | 360000 | 40500 | 7.64 | 63.7% |
| Carbopol | 1103.03 | 370000 | >50000 | 12.00 | 100% |
| Vaseline Aloe | 713.60 | 341000 | 37000 | 8.579 | 71.5% |
| Fresh Moisturizing | | | | | |

Hand and Body

| | | | | | |
|---|---|---|---|---|---|
| Blend 3 | 905.16 | 207000 | 20400 | 6.135 | 51.1% |
| Carbopol | 537.66 | 89000 | 15450 | 10.526 | 87.7% |
| Olay Active | 558.10 | 158000 | 22000 | 10.568 | 88.1% |
| Hydrating Beauty | | | | | |

As the foregoing illustrates, rheological similarities also were observed between personal care product formulations made using the blend of a modified xanthan gum and a galactomannan when compared to personal care products made using Carbopol® or commercial formulations. For example, a Soft-Hold Styling Gel personal care product comprising Blend 3 revealed a hardness of 1450 Pa, a low shear viscosity of 406,000 cP, a high shear viscosity of 49,300 cP, and a shape retention of 73.8%. These rheological properties compared favorably to several rheological properties of a Soft-Hold Styling Gel personal care product comprising Carbopol®. For example, both the high shear viscosity and shape retention showed substantial rheological similarities.

### Sensory Comparisons

To more fully assess the capability of the blends embodied in the present description to serve as a substitute for Carbopol®, the sensory properties of the above-described personal care products were compared to evaluate potential consumer appeal. In particular, the personal care products derived from the described blends comprising a modified xanthan gum and a galactomannan were compared, on the basis of sensory perception, both to similar personal care product formulations that used a comparative amount of Carbopol® and existing commercially available personal care products.

A Quantitative Descriptive Analysis methodology was used, which is well known to those skilled in the art. Panelists were screened in-house and reviewed the definitions of terms for each formulation before evaluation of each formulation. The formulations and the corresponding controls were rated from low (1) to high (5) in each term category and the average score for each category was calculated. For Hair Gel, evaluation was done for clarity (appearance), delivery (easy to squeeze out, integrity of shape, glossy, smooth), pick-up (firm, sticky, cohesive, peaking), smoothness, easy to spread (spreadable, thick, sticky), and rinse-off (easy to rinse from hand, after feel on hand). For Aloe Vera Gel, evaluation was done for clarity (appearance), delivery (easy to squeeze out, integrity of shape, glossy, smooth, stringiness), pick-up (firm, sticky, cohesive, peaking), smoothness, rub-out (wet, spreadable, thick, sticky, easy to absorb, cold), and after feel (sticky, silkiness, heavy skin feel). For Hand and Body Lotion, evaluation was done for delivery (easy to squeeze out, integrity of shape, glossy, smooth), pick-up (firm, sticky, cohesive, stringy, peaking), smoothness, rub-out (spreadable, thick, sticky, easy to absorb), and after feel (sticky, silkiness, heavy skin feel). The results of the sensory evaluations for each personal care product are set forth in the tables below.

### Soft-Hold Styling Gel:

| | Clar- | Deliv- | Pick- | Smooth- | Easy-to- | Rinse- | Over- |
|---|---|---|---|---|---|---|---|
| Sample | ity | ery | Up | ness | spread | off | all |
| Blend 3 | 3.09 | 4.09 | 3.73 | 4.45 | 4.18 | 3.91 | 3.73 |
| Carbopol | 4.73 | 4.27 | 4.27 | 4.64 | 4.45 | 4.45 | 4.50 |
| Pantene | 4.32 | 4.09 | 3.91 | 4.18 | 4.00 | 4.27 | 4.14 |
| Curls | | | | | | | |
| Gel | | | | | | | |

### Aloe Vera Gel:

| | Clar- | Deliv- | Pick- | Smooth- | Rub- | After- | Over- |
|---|---|---|---|---|---|---|---|
| Sample | ity | ery | Up | ness | out | feel | all |
| Blend 3 | 3.25 | 3.38 | 3.50 | 3.75 | 3.50 | 3.31 | 3.25 |
| Carbopol | 4.13 | 4.25 | 4.38 | 4.00 | 3.38 | 3.00 | 3.88 |
| Vaseline | 4.75 | 4.25 | 4.13 | 4.31 | 3.75 | 3.38 | 3.75 |
| Aloe Fresh | | | | | | | |
| Moisturizing | | | | | | | |

### Hand and Body Lotion:

| | | Pick- | | Rub- | After- | |
|---|---|---|---|---|---|---|
| Sample | Delivery | Up | Smoothness | out | feel | Overall |
| Blend 3 | 3.65 | 3.60 | 4.30 | 3.35 | 3.5 | 3.70 |
| Carbopol | 4.40 | 4.40 | 4.50 | 3.85 | 3.70 | 4.10 |
| Olay Active | 4.40 | 4.20 | 4.40 | 3.70 | 3.60 | 4.10 |
| Hydrating | | | | | | |
| Beauty Fluid | | | | | | |

As the foregoing illustrates, the personal care formulations made using a blend that is exemplary of the present description (Blend 3) provided a substantially similar consumer experience to both the personal care products comprising Carbopol® and the commercial products based on the sensory properties. Specifically, the personal care products comprising Blend 3 received overall sensory scores that were between 83% and 90% of the overall sensory scores of the personal care products comprising Carbopol®. Additionally, the personal care products comprising Blend 3 received overall sensory scores that were between 87% and 90% of the overall sensory scores of the commercial personal care products. Most notably, there was not a great deviation in consumer appeal between any of the tested products, suggesting that the “all natural” products comprising the natural polymer blend of a modified xanthan gum and a galactomannan likely will find comparable utility in the marketplace.

It should be apparent that the foregoing relates only to the preferred embodiments of the present invention and that numerous changes and modifications may be made herein without departing from the spirit and the scope of the invention as defined by the following claims and equivalents thereof.

1. A blend for use in a personal care product comprising a modified xanthan gum and a galactomannan, wherein:
the modified xanthan gum comprises a non-pyruvylated xanthan gum, and
the modified xanthan gum and the galactomannan are present in the blend in a ratio from about 1:99 to about 99:1 by weight.

2. The blend of claim 1, wherein the galactomannan is selected from the group consisting of fenugreek gum, guar gum, tara gum, locust bean gum, cassia gum, and combinations thereof.

3. The blend of claim 1, wherein the galactomannan comprises a galactomannan having a low degree of galactose substitution.

4. The blend of claim 3, wherein the galactomannan is selected from the group consisting of locust bean gum, tara gum, cassia gum, and combinations thereof.

5. The blend of claim 3, wherein the modified xanthan gum and the galactomannan are present in the blend in a ratio from about 4:1 to about 99:1 by weight or from about 1:4 to about 1:99 by weight.

6. The blend of claim 3, wherein the modified xanthan gum and the galactomannan are present in the blend in a ratio from about 10:1 to about 99:1 by weight or from about 1:10 to about 1:99 by weight.

7. The blend of claim 1, wherein the galactomannan comprises a galactomannan having a high degree of galactose substitution.

8. The blend of claim 7, wherein the galactomannan is selected from the group consisting of guar gum, fenugreek gum, and combinations thereof.

9. The blend of claim 7, wherein the modified xanthan gum and the galactomannan are present in the blend in a ratio from about 1:9 to about 9:1 by weight.

10. The blend of claim 7, wherein the modified xanthan gum and the galactomannan are present in the blend in a ratio from about 1:5 to about 5:1 by weight.

11. The blend of claim 7, wherein the modified xanthan gum and the galactomannan are present in the blend in a ratio from about 1:2 to about 2:1 by weight.

12. A method for preparing a blend for use in a personal care product comprising combining a modified xanthan gum and a galactomannan to form a blend, wherein the modified xanthan gum comprises a non-pyruvylated xanthan gum, and wherein the modified xanthan gum and the galactomannan are present in the blend in a ratio from about 1:99 to about 99:1 by weight.

13. The method of claim 12, wherein the galactomannan is fenugreek gum, guar gum, tara gum, locust bean gum, cassia gum, or combinations thereof.

14. The method of claim 12, wherein the galactomannan comprises a galactomannan having a low degree of galactose substitution and wherein the modified xanthan gum and the galactomannan are present in the blend in a ratio from about 4:1 to about 99:1 by weight or from about 1:4 to about 1:99 by weight.

15. The method of claim 12, wherein the galactomannan comprises a galactomannan having a high degree of galactose substitution and wherein the modified xanthan gum and the galactomannan are present in the blend in a ratio from about 1:9 to about 9:1 by weight.

16. The method of claim 12, wherein the modified xanthan gum and the galactomannan are combined as individual dry powders and mixed into a homogenous dry powder blend.

17. A personal care product comprising a blend of a modified xanthan gum and a galactomannan, wherein:
the modified xanthan gum comprises a non-pyruvylated xanthan gum; and
the modified xanthan gum and the galactomannan are present in the blend in a ratio from about 1:99 to about 99:1 by weight.

18. The personal care product of claim 17, wherein the blend is present in the personal care product in an amount from about 0.1% to about 4.0% by weight of the personal care product.

19. The personal care product of claim 17, wherein the galactomannan is selected from the group consisting of fenugreek gum, guar gum, tara gum, locust bean gum, cassia gum, and combinations thereof.

20. The personal care product of claim 17, wherein the galactomannan comprises a galactomannan having a low degree of galactose substitution and wherein the modified xanthan gum and the galactomannan are present in the blend in a ratio from about 4:1 to about 99:1 by weight or from about 1:4 to about 1:99 by weight.

21. The personal care product of claim 17, wherein the galactomannan comprises a galactomannan having a high degree of galactose substitution and wherein the modified xanthan gum and the galactomannan are present in the blend in a ratio from about 1:9 to about 9:1 by weight.

22. The personal care product of claim 17, wherein the personal care product is a styling gel, a moisturizer, a lotion, a deodorant, a toothpaste, a body wash, a bath gel, a body gel, a hand sanitizer, a shampoo, a conditioner, or combinations thereof.
